(19) United States
(12) Patent Application Publication
(10) Pub. No.: US 2017/0224866 A1
(43) Pub. Date: Aug. 10, 2017

(54) WOUND CARE PRODUCT

(71) Applicant: PAUL HARTMANN AG, Heidenheim (DE)

(72) Inventors: Pierre CROIZAT, Herbrectingen (DE); Martin JUNGINGER, Hermaringen (DE); Sandra STELZER, Heidenheim (DE)

(21) Appl. No.: 15/497,345

(22) Filed: Apr. 26, 2017

Related U.S. Application Data
(62) Division of application No. 14/123,894, filed on Dec. 4, 2013.

(30) Foreign Application Priority Data
Jun. 30, 2011 (DE) 10 2011 106 046.8

(51) Int. Cl. A61L 15/42; A61L 15/26; A61L 15/20; A61F 13/00; A61F 13/02

(52) U.S. Cl. CPC A61L 15/425; A61F 13/00017; A61F 13/023; A61F 13/00063; A61F 13/00029; A61L 15/20; A61L 15/26

(57) ABSTRACT

A wound care product having buffer substances and a wound cover, and a method for treatment of a wound therewith. The wound cover has a foam. The pH value of the buffer solution obtained when the buffer substances are dissolved in demineralized water at 37° C. is between 3 and 7.

This is a divisional of application Ser. No. 14/123,894 which is the national stage of international application PCT/EP2012/002743, filed Jun. 29, 2012, the entire disclosure of which is hereby incorporated by reference herein.

The present invention relates to a wound care product, especially for the moist treatment of wounds during the inflammatory phase and the granulation phase of wound healing.

### BACKGROUND OF THE INVENTION

The healing of skin wounds is based on the ability of the skin, epithelium, and connective and supporting tissue to regenerate. The regeneration itself is characterized by a complex occurrence of overlapping cellular activities which advance the healing process step by step. Thus, the literature describes three essential wound healing phases, especially in the case of wounds with loss of tissue. These include the inflammatory or exudative phase for hemostasis and wound cleansing (phase 1, cleansing phase), the proliferative phase for construction of granulation tissue (phase 2, granulation phase) and the differentiation phase for epithelialization and scarring (phase 3, epithelialization phase). It has been found that wound healing is especially promoted by modern, moist wound treatment. As part of moist wound treatment, use is made of, inter alia, wound dressings having a foam layer. Such a foam layer provides growing tissue with a matrix which stimulates wound healing and can simultaneously take up and bind relatively large amounts of wound exudate. It has also proven effective to use hydrogels for maintaining the moistness of wounds, the hydrogels additionally reducing skin irritations at the wound edge.

It has been known for a long time that disturbances can occur during wound healing, especially during the inflammatory phase and the granulation phase of wound healing. In the context of the present invention, the term “wound” includes the wound base. Wound exudate can be found in the wound. Wound healing can in this case be influenced by the pH of a wound or by the pH of the wound exudate present in the wound. Healthy skin usually has a pH in the acidic range, which is approximately between pH 4.0 and pH 5.7.

In the case of wounds having disturbed wound healing, more particularly chronic wounds, an alkaline pH of the wound or the wound exudate can be frequently observed. Measurement can be carried out by, for example, an in vitro measurement of the pH of wound exudate. A shift in pH from the acidic range to the alkaline range can, for example, be caused by the propagation of bacteria or by the formation of necrotic tissue.

Necrotic tissue and pathological microorganisms can act on the physiological metabolism while wound healing is taking place. This frequently leads to local hypoxia, and then to further degradation of surrounding tissue.

The resultant alkaline environment can favor further tissue-degrading processes. In addition, the alkaline environment can stimulate the propagation of further pathogenic microorganisms and thus additionally prevent wound healing. In the context of this invention, chronic wounds mean wounds which do not heal within an expected period of from 4 to 6 weeks.

Numerous wound pH-modifying products are known in the prior art. U.S. Pat. No. 4,813,942 describes a wound dressing having an adhesive layer which comprises polyisobutylene and a hydrocolloid and can adjust the pH of the wound to the range from pH 4.8 to pH 6.5. EP1322348 likewise describes a wound pH-lowering adhesive, which can be provided with, inter alia, citric acid and inorganic buffers. EP0506300 describes a wound bandage comprising a hydrophilic gel which comprises polyurethane and poly(N-vinyl lactam) having a K-value of greater than 60 and more than 1.4 mole equivalents of acid groups. WO01/39582 describes a wound pH-influencing buffer system within the pH range of from 2 to 5.5, which is especially effective against the yeast fungus Candida albicans. The buffer system is used in this case in a skin-covering article such as a wound dressing, a diaper or a mucous membrane-covering article, and preferably comprises a superabsorbent having pH-stabilizing properties. EP1341561 describes a multilayered wound bandage material which comprises a hydrogel layer and a barrier layer, the barrier layer comprising a material which is pH-dependent with respect to solubility. The material is insoluble at pH 4, but water-soluble at pH 8. Thus, the barrier layer becomes permeable at an alkaline pH in order to take up the accruing wound secretion in a further layer. The hydrogel facing the wound contains a weak, water-soluble buffer system.

### SUMMARY OF THE INVENTION

It is an object of the present invention to provide an improved wound care product; more particularly, chronic wound healing shall be improved and the disadvantages of the prior art shall be overcome. Proceeding from commercially available wound dressings, it is a further object of the present invention to provide an improved wound dressing which has high absorption ability with respect to wound exudate and, more particularly, can be used for wound healing in the exudative phase or the granulation phase. Furthermore, a wound dressing shall be provided which influences the pathological state of a wound in such a way that it is possible for a wound healing process to take place rapidly and with low scarring. To this end, the wound dressing shall have in particular good absorption power.

To achieve this object, the wound care product according to the invention comprises buffer substances and a wound dressing, the wound dressing comprising a foam. The wound care product is notable for the fact that the pH of the buffer solution resulting upon dissolution of the buffer substances in demineralized water at 37° C. is between pH 3 and pH 7. The wound dressing can be single-layered or multilayered.

The invention further provides methods for producing a wound care product.

The invention further comprises the treatment of wounds, more particularly chronic wounds, using the wound care product according to the invention.

### BRIEF DESCRIPTION OF THE DRAWINGS

FIG. 1: shows a cross section through a multilayered wound care product

FIG. 2: shows a cross section through a further embodiment of a multilayered wound care product

FIG. 3: shows a top view of the side of the multilayered wound care product from FIG. 1 that is facing the wound during use

### DETAILED DESCRIPTION OF THE INVENTION

In the context of the invention, the term “buffer solution” refers to a composition comprising a weak acid and its corresponding base or comprising a weak base and its corresponding acid. The counterions of the corresponding base can, for example, be sodium ions. The buffer solution can in particular consist of a weak acid and the salt of said acid. The composition is present in an aqueous solution. The individual substances of the particular composition, i.e., acid and base or salt, are referred to in this case as buffer substances. The buffer substances are preferably nontoxic, skin-friendly and physiologically safe substances. The pH of the buffer solution is a result of the protolytic equilibrium of the buffer pair of acid and base or salt. The pH of a buffer solution can be calculated in good approximation according to the Henderson-Hasselbalch equation. Said equation states that the pH of a buffer solution is equal to the pKa of the acid component of the buffer solution plus the common logarithm of the quotient arising from division of the concentration of the corresponding base by the acid concentration. Consequently, it follows from the definition of the Henderson-Hasselbalch equation that the pH is equal to the pKa when the buffer substances have identical concentrations. In the context of the present invention, the use of identical concentrations of the respective buffer substances is preferred. In such cases, the pH of the buffer solution resulting upon dissolution of the buffer substances corresponds, in good approximation, to the pKa of the acid component of the buffer substances. Thus, the Henderson-Hasselbalch equation can be utilized to estimate the pH of the resulting buffer solution by means of the pKa of buffer substances.

In the case of a wound care product according to the invention, the pH of the buffer solution resulting upon dissolution of the buffer substances is, however, not calculated, but determined by means of pH measurement. For such a measurement, the buffer substances are completely dissolved at a concentration of 0.1 M in one liter of demineralized water at a temperature of 37° C. with stirring to produce a buffer solution. Stirring of the solution can be carried out using, for example, a commercially available magnetic stirrer. The pH of the buffer solution can be measured in a customary manner per se using a commercially available potentiometry-based pH meter. The pH meter should be calibrated before the measurement using commercially available calibration solutions and be used according to the operation instructions from the manufacturer. The pH of the buffer solution is measured by dipping the measurement electrode of the pH meter into the buffer solution while the buffer solution is being stirred. Subsequently, the pH measured is read off from the display of the pH meter.

Such a buffer solution is suitable for stabilizing the pH of a liquid within a particular pH range, said pH range being referred to as buffer range in the context of the present invention. Said liquid can be wound exudate.

The buffer range of a buffer solution is defined in a customary manner per se as a pH range having a lower limit and an upper limit. The lower limit of the buffer range is, in the aforementioned case of identical concentrations of the buffer substances, the pKa of the buffer solution resulting upon dissolution of the buffer substances minus one pH unit. The upper limit of the buffer range is, in the aforementioned case of identical concentrations of the buffer substances, the pKa of the aforementioned buffer solution plus one pH unit.

In addition, buffer solutions have the property of stabilizing the pH within the pH range for a prolonged period in the case of addition of a liquid having a pH different to the pH of the buffer solution. A quantitative measure of the aforementioned pH-stabilizing property is the buffering capacity of the buffer solution.

In the context of the present invention, the buffering capacity is defined by the amount in moles of NaOH which is required in titration experiments to alter by one pH unit the pH of a liquid amount of 15 ml of demineralized water per g of product containing buffer substances. The amount of NaOH is determined via the consumption of 0.1 M NaOH and reported in moles.

In one embodiment of the invention, the wound care product comprises buffer substances, wherein the pH of the buffer solution resulting upon dissolution of the buffer substances in demineralized water at 37° C. is between pH 3.5 and pH 6. This is advantageous because said pH values are within the physiological pH range of wounds which are healing well.

In a further embodiment of the invention, the wound care product comprises buffer substances, wherein the pH of the buffer solution resulting upon dissolution of the buffer substances in demineralized water at 37° C. is between pH 3.8 and pH 5. This is advantageous because some buffer substances which are simple and cost-effective to produce, for example benzoic acid/benzoate (pKa 4.2) or lactic acid/lactate (pKa 3.9), have pKa values within said pH range.

In a further preferred embodiment, the foam component of the wound dressing comprises the buffer substances, wherein the buffer substances are present especially in the polymer matrix of the foam. The buffer substances are first dissolved through the absorption of wound exudate. The buffer solution can then diffuse back into the wound and stabilize the pH there within the acidic range. Since the pH of the liquid absorbed into the foam is also stabilized within the acidic pH range, the propagation of germs, which prefer an alkaline environment, can be prevented. Thus, back-contamination of the wound with germs is prevented and the intervals for bandage changes can be prolonged if needed.

In a further development of the wound care product according to the invention, the buffer substances are distributed homogeneously in the foam.

In one embodiment of the wound care product according to the invention, it is advantageous for the buffering capacity of the wound care product to be, at a concentration of the buffer substances of 0.05 M, at least 0.25 mol of NaOH, more particularly at least 0.55 mol of NaOH.

In a further embodiment, the wound care product according to the invention comprises an additional layer comprising a hydrogel. More particularly, the additional layer comprising a hydrogel is arranged between wound and foam during use. Preferably, the layer comprising a hydrogel is in direct contact with the wound surface during use, i.e., the hydrogel layer serves as wound contact layer.

In a preferred embodiment, a hydrogel serving as wound contact layer comprises buffer substances. The buffer substances present in the gel can be dissolved in the wound exudate. The resulting buffer solution can diffuse into the wound and stabilize the pH there within the acidic range. Since the liquid absorbed into the foam is also buffered within the acidic pH range, the propagation of germs, which prefer an alkaline environment, is also prevented in this variant. Thus, back-contamination of the wound with germs is prevented and the intervals for bandage changes can be prolonged if needed. Furthermore, the hydrogel can reduce skin irritations. In addition, the gel provides dry wounds in particular with sufficient moisture. This is advantageous in the case of use with a dry foam wound dressing. The hydrogel containing buffer substances can be combined with a foam wound dressing containing buffer substances. This combination ensures an especially high buffering capacity and constant moisturization of the wound.

In a further preferred embodiment, the wound care product according to the invention comprises buffer substances, wherein the buffering capacity of the wound care product is, at a concentration of the buffer substances of 0.05 M, at least 0.08 mol of NaOH, more particularly at least 0.1 mol of NaOH.

In a particularly preferred embodiment of the invention, the wound care product comprises a buffer solution, wherein the buffer solution comprises citric acid and citrate as buffer substances. Said buffer solution is advantageous because it is especially cost-effective to produce.

In another embodiment of the invention, the wound care product comprises the buffer substances lactic acid and lactate. This buffer system is advantageous because, with a pKa of 3.9, it can stabilize an especially low pH. Furthermore, lactic acid and lactate have been used for a long time in the food industry, since they are simple to chemically synthesize and are especially well-tolerated from a biological point of view.

In a further embodiment of the invention, the wound care product comprises benzoic acid and benzoate as buffer substances. This buffer solution is advantageous because, with a pKa of 4.2, it can stabilize a pH which is especially advantageous, from a physiological point of view, for normal wound healing.

In a preferred embodiment of the invention, the buffering capacity of the wound care product reaches 90% of the maximum buffering capacity measurable after 24 hours after no later than three hours. Said rapid dissolution of the buffer substances is especially advantageous because the wound care product can rapidly provide a high buffering capacity when used on a wound. Rapidly providing a high buffering capacity makes it possible for the pH of wound exudate situated in the wound to be reliably stabilized within the acidic pH range from the start of treatment.

The invention further provides methods for producing a wound care product. The method for producing a wound care product comprises providing a foam, providing a buffer solution containing buffer substances, the pH of the buffer solution at 37° C. being between pH 3 and pH 7. The method further comprises providing further layers. Subsequently, the foam is impregnated with the buffer solution and subsequently either completely or partially dried. For the purposes of impregnation, the foam is immersed for at least 60 seconds in a buffer substance-containing buffer solution (15 ml/g of foam) which contains buffer substances having a total concentration of from 0.01 to 0.6 M. In the context of the present invention, the term “concentration” refers to, in each case, the total concentration of all buffer substances used. Thereafter, the foam is drip-dried and subsequently completely dried for three days under a standard atmosphere (23° celsius, 50% rel. humidity, see DIN EN ISO 139). For partial drying of the foam, a shorter drying time can be selected. After the impregnation of the foam with the buffer solution, the method further comprises the joining of the impregnated foam and the further layers to form a multilayered wound dressing. This method is especially suitable for giving pH-changing properties to a foam material after its production.

In a further conceivable embodiment, the wound care product can be impregnated with a buffer solution directly before use on the wound so that it obtains pH-stabilizing properties. It would likewise be conceivable and advantageous to add the buffer substances as early as during the production of the foam.

It has been found that, when using the buffer solutions citric acid/citrate buffer, benzoic acid/benzoate and lactic acid/lactate, a concentration of from 0.03 M to 0.6 M buffer substances is especially advantageous for improved wound healing. For the impregnation of the foam, preferably 15 ml of the buffer solution are used per g of foam. This amount of liquid corresponds to the uptake capacity of a hydrophilic polyurethane foam having a density of from 70 to 110 kg/m³, making it possible to ensure an almost complete uptake of the buffer solution by the foam. Foams which differ in density from the range described would be able to be impregnated according to their uptake capacity.

In a further advantageous embodiment, the invention provides a method for producing a wound care product in which the buffer substances are present in a hydrogel. The method for producing a wound care product comprises providing a hydrogel, providing a wound dressing comprising a foam, and providing a buffer solution containing buffer substances, the pH of the buffer solution at 37° C. being between pH 3 and pH 7. The method further comprises providing further layers. The buffer substances are dissolved in demineralized water and mixed with the gel, yielding a concentration of from 0.01 to 0.6 M buffer substances for the mixture. The method further comprises joining the wound dressing and the hydrogel, the gel forming the wound contact layer of the wound care product.

The invention further comprises the treatment of wounds, more particularly chronic wounds, using the wound care product according to the invention. More particularly, it is possible to treat wounds which occur in traumatic injuries with loss of tissue. It is further possible to use the wound care product according to the invention to treat ulcer wounds such as, for example, decubitus ulcer wounds and wounds arising due to venous insufficiency. The wound care product according to the invention is also suitable for use in treating thermal and chemical wounds.

It has been found that wound healing can be influenced in an especially advantageous manner when using the wound care product according to the invention. The advantageous effect when using the wound care product according to the invention in wound therapy arises especially when treating chronic wounds. When using the wound care product according to the invention for wound therapy, the occurrence of wound healing disturbances can be reduced and scarring can be minimized. In this case, the wound healing-promoting properties, known per se, of foam wound dressings appear to work together with the pH-stabilizing properties of the buffer substances in a mutually reinforcing manner.

Generally, it is desirable for a wound bandage component making direct contact with the wound base to have a long residence time on the wound, since each bandage change can disturb the wound healing process. Furthermore, bandage change is unpleasant for the patient and increases the effort for the attending medical personnel. Wound dressings having foams are suited to long residence times on the wound because they have high absorption power for wound exudate.

If a wound dressing known from the prior art comprising a foam as wound contact layer remains for a few days on the wound, a shift in the pH of wound and wound exudate into the alkaline pH range can, however, be frequently observed.

It has been found that the wound care product according to the invention, compared to conventional wound dressings comprising foam, can remain on the wound for at least just as long, for example more than two days. This avoids additional bandage changes, which might otherwise impair wound healing. It was possible to observe that the buffering effect remains largely preserved over the residence time of the wound care product on the wound.

It was further observed that the wound care product according to the invention allows especially effective microdebridement. Microdebridement refers to the gentle removal of wound exudate and destroyed tissue from the wound during bandage change. Microdebridement supports rapid wound healing.

The use of the wound care product according to the invention is indicated especially in the case of strongly exudative wounds in the inflammatory phase or the granulation phase of wound healing, more particularly in the case of secondary healing wounds. It has been found that the pH-stabilizing properties of the wound care product can facilitate the detachment of necrotic tissue and can minimize the spread of germs in the wound, improving the wound healing-promoting effect of a foam wound dressing in a mutually reinforcing manner. For wound treatment using the present wound care product, an interval of about from 1 to 3 days for a bandage change is advisable, depending on the strength of wound exudation. This is because it has been found that microdebridement at said interval can take place especially gently for the wound without expecting already strong adhesions with the foam layer, which could traumatize the wound again.

A wound care product according to the invention preferably comprises a hydrophilic polyurethane foam. The use of a hydrophilic polyurethane foam is advantageous for rapid wound healing because such foams have a high absorption capacity and are therefore preferably used in the cleansing phase of wound healing in the case of strong exudation. A further advantage of polyurethane foams is that only slight shear forces are exerted on a wound to be treated and the wound is thus well cushioned.

In connection with the present invention, a hydrophilic polyurethane foam is understood to mean a polyurethane foam which can take up a liquid into its polyurethane matrix and into its pores and store it, i.e., absorb it, and can release at least some of the liquid taken up. Suitable in this case as hydrophilic polymer foams are in particular open-pore, hydrophilic polyurethane foams. Accordingly, a particularly preferred wound dressing comprises a layer comprising an open-pore, hydrophilic polyurethane foam. According to the invention, preference shall be given to using polyurethane foams having a high absorption capacity for liquids of more than 2.5 g, preferably more than 10 g, even more preferably more than 16 g, of isotonic saline solution per gram of foam polymer. The absorption ability is determined in accordance with DIN EN 13726-1:2002 (3 min measurement). Such a foam can absorb and reliably trap germs and cell debris, but while doing so can nonetheless lie on the wound in a soft and supple manner with a good cushioning effect.

Preferably, the hydrophilic polyurethane foam has a mean pore size of less than 1000 μm, more particularly from 100 to 1000 μm, preferably from 100 to 500 μm, and very particularly preferably from 100 to 300 μm. The preferred method for determining pore size is the measurement of the diameter of a multiplicity of pores on a sectional plane oriented in parallel to the wound contact side of the foam layer or of the wound care product. Pore size can be measured by viewing the pores in a light or electron microscope and comparing the pore diameter with a suitable scale. The foam can have a homogeneous pore size or a pore size gradient across the thickness of the foam layer. When using a foam having a pore size gradient, efficient drainage of wound exudate is ensured by a reduction in pore size, starting from the wound contact layer, from relatively large pores on the wound contact side (mean pore size 200-300 μm for example) through to relatively small pores on the foam side facing away from the wound during use (mean pore size 100-200 μm for example). Efficient drainage of wound exudate occurs because a capillary effect can be generated for especially good absorption of liquids. At the same time, the foam can provide a sufficient amount of moisture for a wound. A foam having a pore size gradient across the thickness of the foam and a pore size of less than 1000 μm is used, for example, in the product Permafoam from Paul Hartmann AG. Furthermore, it is especially advantageous for the wound dressing to also have a water vapor-permeable polyurethane cover layer. It is further advantageous for the water vapor-permeable polyurethane cover layer to have a water vapor permeability (“upright”, measured in accordance with DIN EN 13726-2 at a temperature of 37° C.) of more than 600 g/m² in 24 h.

Furthermore, it is conceivable and advantageous for the wound dressing to have a reticular hydrogel on the wound side. It is further advantageous for the wound dressing to comprise a polyurethane cover layer on the side facing away from the wound.

In addition, it is advantageous for the foam to have a density of from 70 to 110 kg/m³. In a further embodiment of the invention, a hydrophobic PU foam having a density of from 10 to 50 kg/m³ can be used. Such foams are used especially in the case of wound dressings intended for negative-pressure wound therapy. In a further embodiment of the invention, it would be further conceivable and advantageous to use silicone foams having a density of up to 300 kg/m³.

Polyurethane foam materials are usually obtainable by reacting a curable mixture, comprising the components polyisocyanate and isocyanate-reactive compounds, more particularly polyol, and also catalysts, blowing agents and optionally additives. Isocyanates which can be used are generally known as aliphatic, cycloaliphatic and/or, in particular, aromatic polyisocyanates. Suitable for preparing polyurethanes are, for example, diphenylmethane diisocyanate, here more particularly 4,4′-diphenylmethane diisocyanate, mixtures composed of monomeric diphenylmethane diisocyanates and higher polycyclic homologs of the diphenylmethane diisocyanate, tetramethylene diisocyanate, hexamethylene diisocyanate, toluene diisocyanate or mixtures thereof. Isocyanate-reactive compounds used are usually polyols such as polyetherols and/or polyesterols.

In addition, foam wound dressings comprising a polyurethane foam having a layer thickness of from 0.1 cm to 1.8 cm, preferably from 0.3 cm to 1.5 cm, and very particularly preferably from 0.5 cm to 1.0 cm have been found to be especially advantageous embodiments. The layer thickness can be identical at every position of the wound contact layer or can assume different values in different regions of the wound contact layer. More particularly, it is also envisaged that the absorbent layer or the polyurethane foam has tapered edges.

Preferably, the wound care product has a substantially rectangular basic form. In this case, particular preference is given to a size range of from 8 cm×8 cm to 20 cm×20 cm. The thickness of the wound care product is preferably less than 2 cm, the foam layer preferably having a thickness between 0.1 cm and 1.8 cm.

According to the present invention, an additional material can be used as wound contact layer. Here, a wound contact layer is in direct contact with the wound when using the wound dressing according to the invention. The wound contact layer can be used solely to space the foam from the wound to be treated. The additional layer has the advantage of ensuring that the wound care product is detached during a bandage change in a manner that is especially gentle to tissue. The wound contact layer can exert further functions with regard to the wound to be treated. For example, the wound contact layer can provide the wound with moisture, have wound edge-maintaining properties, reduce skin irritations, or have an antiadherent effect.

A wound dressing according to the invention can comprise a wound contact layer comprising a hydrogel, a polymer film, a hydrocolloid matrix, a polymer network, a nonwoven and/or an adhesive. In the context of the invention, the term “hydrogel” or “gel” refers to a finely dispersed system composed of at least one solid phase and one liquid phase. Said solid phase forms a spongy, three-dimensional network having pores filled by a liquid (lyogel) or else a gas (xerogel). Both phases permeate each other completely.

Such a hydrogel is preferably an amorphous hydrogel comprising in particular the components sodium chloride, potassium chloride, calcium chloride dihydrate, Carbopol, glycerol, Natrosol and Blanose. The components of the gel are mixed with an aqueous buffer solution which can buffer the pH within the acidic range. Preferably, said aqueous buffer solution comprises equimolar concentrations of the buffer substances, i.e., the same molar proportion of acid and base.

A further advantageous recipe for a hydrogel comprises 20-60 percent by weight of propylene glycol, 3-percent by weight of a polyethylene oxide-based diamine, from 0.5 to 1.5 percent by weight of NaCl, 5-percent by weight of isocyanate, said substances being present in an aqueous buffer solution which can buffer the pH within the acidic range. Preferably, said aqueous buffer solution comprises equimolar concentrations of the buffer substances, i.e., the same molar proportion of acid and base.

The hydrogel can be used in different ways for wound treatment. The gel can first be applied to the wound and then be covered using a wound dressing. Another way of using the gel for wound treatment consists in using a wound dressing holding ready the hydrogel in a wound contact layer. By this means, the properties of the gel with respect to stabilizing the pH within the acidic range are provided in the wound.

The buffer substances envisaged in the context of the invention preferably have pKa values between 3.5 and 6.9. Examples of such buffer substances are acetic acid/acetate (pKa 4.76), benzoic acid/benzoate (pKa 4.2) or lactic acid/lactate (pKa 3.9). The advantage of said buffer systems is, in particular, that they are used in numerous products of the food industry and are therefore well established. In addition, they can be synthesized chemically in a simple and cost-effective manner. If the invention is carried out in such a way that the foam layer comprises the buffer substances, a buffer solution arises through the action of wound exudate on the foam because the buffer substances are dissolved in the wound exudate and can stabilize the pH of this solution and of the wound within the acidic pH range.

When using the wound care product according to the invention, particularly the exudation of the wound provides for effective dissolution and distribution of the buffer substances in the wound.

Embodiments of wound care products according to the invention will now be more particularly elucidated with reference to drawings. However, the invention is not to be understood to be reduced to the embodiments depicted in the drawings or in the description of the drawing.

On the contrary, the device according to the invention also encompasses combinations of the individual features of the alternative forms. In the figures:

| Reference sign | Meaning |
|---|---|
| 10 | Wound care product |
| 11 | Polyurethane foam |
| 12 | Polyurethane cover layer |
| 13 | Adhesive |
| 15 | Hydrogel |
| 16 | Adhesive edge |
| 20 | Wound care product comprising hydrogel |

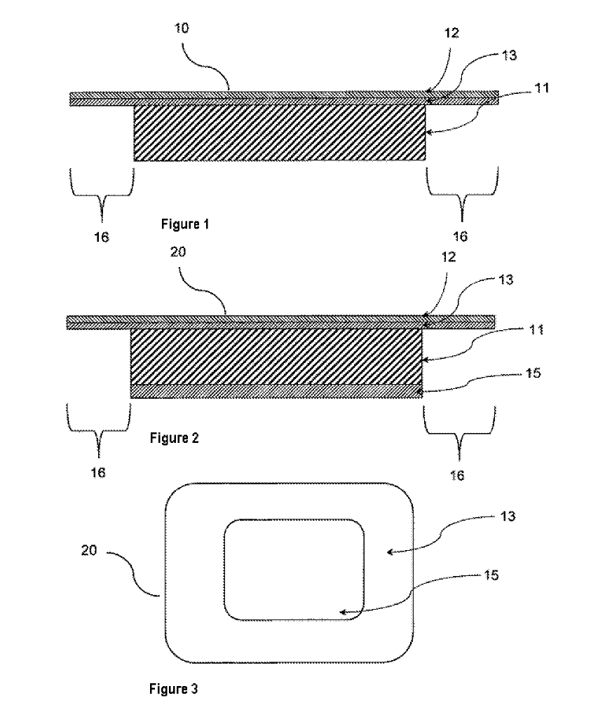

FIG. 1:

The embodiment of the invention shown diagrammatically in FIG. 1 comprises the wound care product 10, a multilayered wound dressing. Said wound dressing comprises an elastic, water vapor-permeable cover layer 12 composed of polyurethane as support material, said cover layer being provided with an acrylate adhesive 13 on one side. The polyurethane cover layer 12 protrudes beyond a soft, hydrophilic polyurethane foam 11 and thus forms an adhesive edge 16 which can be used to fix the product on the skin of the patient. The polyurethane cover layer 12 preferably has dimensions of 15 cm×15 cm, and in the center on the side of the polyurethane cover layer 12 that is provided with acrylate adhesive 13, the polyurethane foam 11 is provided with a size of 10×10 cm.

The polyurethane foam 11 has a density of from 70 to 110 kg/m³. The pore size of the foam has a gradient with increasingly smaller pores from the wound side toward the side of the foam layer that is facing away from the wound and is, on average, from 100 to 300 μm. The polyurethane foam 11 was provided, by impregnation, with the buffer substances benzoic acid and benzoate (concentration 0.04 M) in order to achieve stabilization of the pH within the pH range of from pH 4 to pH 4.5 and a buffering capacity of about 0.3-0.4 mol of NaOH. After impregnation, complete drying of the polyurethane foam 11 was performed, and so the wound care product 10 is present in dry form before use.

It would also be conceivable and advantageous to prepare moist foams having buffer properties, which are especially suitable for treating dry wounds. In one variant of the wound care product 10, the polyurethane foam 11 is not completely dried after impregnation, but instead still has buffer solution in the polymer matrix of the foam layer after the drying operation. In this case, a shorter period for the drying of the foam can be envisaged after impregnation, resulting in the foam having residual moisture. The residual moisture of the polyurethane foam 11 is preferably between 4 g and 12 g of liquid per g of foam. Residual moisture of from 4 g to 12 g of liquid per g of foam corresponds approximately to the amount of liquid which a hydrophilic polyurethane foam 11 can retain in its polymer matrix by swelling. A polyurethane foam 11 having residual moisture of from 4 g to 12 g of water per g of foam contains little liquid, and so the polyurethane foam 11 continues to retain good absorption abilities. A wound care product 10 comprising a polyurethane foam 11 having residual moisture provides a wound with moisture from the start of treatment. In addition, said wound care product comprises an absorbent polyurethane foam 11 having a sufficient absorption capacity. Absorption is achieved by the polyurethane foam 11 soaking up wound exudate into its pores. In this regard, it has been found to be particularly advantageous for the polyurethane foam 11 to be able to generate a capillary effect. For example, a capillary effect can be generated by means of a pore size gradient. Such a pore size gradient arises through a mean pore size which decreases from one side of the polyurethane foam layer 11 to the other side of the polyurethane foam layer 11. Preference is given to the side facing the wound during use having larger pores than the side facing away from the wound. The absorption of wound exudate withdraws substances such as, for example, wound tissue degradation products or bacterial cytotoxic excretions, which may possibly adversely affect wound healing, from the wound surface. At the same time, moisture is provided in a sufficient amount.

Polyurethane foams 11 having residual moisture have a lower absorption capacity compared to dry foams. Dry polyurethane foams 11, especially when no releasable buffer substances are present, can rapidly take up large amounts of wound exudate. The rapid absorption of wound exudate can lead to a dry wound surface. Dry wound surfaces impede a rapid wound healing process and can favor scarring. Thus, wound care products 10 having moist wound dressings are outstandingly suitable for use in the epithelialization phase or granulation phase of wound healing. The wound dressing 10 according to the invention can thereby particularly promote the granulation and/or the epithelialization of the wound in a natural manner.

FIG. 2:

In the embodiment of the invention shown in FIG. 2, the wound care product 20 comprises a hydrogel 15 which is arranged between wound and polyurethane foam 11. Said hydrogel 15 has a total concentration of 0.05 M citric acid and citrate as buffer substances. The hydrogel 15 is suitable for stabilizing the pH of a wound within the range between pH 4 and pH 6. The wound care product 20 has a buffering capacity of 0.1-0.2 mol of NaOH. It is advantageous for the hydrogel 15 to comprise 10-20 percent by weight of glycerol, 0.5-3 percent by weight of hydroxycellulose and 0.2-2 percent by weight of sodium chloride. The polyurethane cover layer 12 has dimensions of 11 cm×11 cm, and a foam layer composed of soft, hydrophilic polyurethane 11 having a size of 6×6 cm is situated in the center on the side of the polyurethane cover layer 12 that is provided with acrylate adhesive 13.

In one embodiment of the wound care product 20, a hydrogel 15 comprising 20-60 percent by weight of propylene glycol, 3-10 percent by weight of a polyethylene oxide-based diamine, from 0.5 to 1.5 percent by weight of NaCl and 5-15 percent by weight of isocyanate is used.

In a further embodiment of the wound care product 20, the hydrogel 15 comprises the buffer substances lactic acid and lactate in a total concentration of 0.05 M and can buffer the pH within the pH range of pH 3.5 to pH 4.2 with a buffering capacity of approximately 0.1-0.15 mol of NaOH.

In a further development of the wound care product 20 shown in FIG. 2, it would be possible for the polyurethane foam 11 and the hydrogel 15 to contain buffer substances. Through the combination of a polyurethane foam 11 and a hydrogel 15, with both the polyurethane foam 11 and the hydrogel 15 comprising buffer substances for the stabilization of the pH of a wound within the acidic pH range, it is possible to achieve an especially high buffering capacity. Thus, the exemplary embodiment described here is especially suitable for strongly exudative wounds, the pH of which might increase into the alkaline pH range owing to inflammatory processes and/or a bacterial infection.

FIG. 3:

The wound side of the wound care product 20 from FIG. 2 is shown in top view.

Experiments relating to the pH-stabilizing properties of wound care products according to the invention will now be elucidated.

In the case of a wound care product according to the invention comprising buffer substances and a wound dressing having a foam layer, it is possible to measure the release of buffer substances into the wound via a simple test system by means of in vitro titration experiments. The ability of the wound care product according to the invention to stabilize a pH within an acidic pH range is described by the buffering capacity. The higher the buffering capacity, the more wound exudate that can be stabilized within the acidic pH range by the wound care product according to the invention.

To measure the buffering capacity of the foam present in the wound care product, 1 g of the foam layer of the wound care product according to the invention was placed in a water bath containing 15 ml of demineralized water. The water bath containing the wound care product was shaken on a shaker at 100 mpm (shaking movements per minute) for 24 hours at room temperature (25° C.). During the 24 hours, the buffer substances were able to dissolve in the demineralized water from the foam layer and form a liquid having pH-stabilizing properties. Subsequently, the liquid, which contained buffer substances, was automatically titrated together with the product. Through the consumption of NaOH, it was possible to determine the buffering capacity. Triplicate measurements were carried out in each case. It was possible to observe that the pH-stabilizing effect was largely preserved over the 24-hour experimental period.

If the hydrogel component contained in the wound care product was present, its buffering capacity was determined analogously, i.e., 1 g of the gel layer was placed in a water bath containing 15 ml of demineralized water. The release of the buffer substances from the gel layer and the determination of the buffering capacity was carried out as described above for the foam layer.

In preliminary experiments, buffer substance concentrations of from 0.01 M to 0.6 M were found to be advantageous for stabilizing the pH within the acidic pH range while being gentle to tissue. It was found that a polyurethane foam (PermaFoam from Paul Hartmann AG) had an especially high buffering capacity of (0.72+/−0.05) mol of NaOH (see table 1) when the buffer substances citric acid and citrate (concentration 0.05 M) were used for impregnation. The buffer substances benzoic acid and benzoate (concentration 0.04 M) had a lower buffering capacity of (0.33+/−0.02) mol of NaOH. The buffer substances lactic acid/lactate (concentration 0.05 M) likewise had a lower buffering capacity of 0.30 mol+/−0.01 mol of NaOH.

TABLE 1
Buffering capacity of foam layers impregnated
with buffer substances and gels admixed with buffer
substances according to the buffer substances used and
the concentration of the buffer substances. σ:
empirical standard deviation.

| | | Concentration | | |
|---|---|---|---|---|
| | | of | Buffering | |
| | | the buffer | capacity | +/− σ |
| Buffering | Buffer | substances | [mol | [mol |
| layer | substances | [M] | NaOH] | NaOH] |
| Polyurethane | Citric | 0.05 | 0.72 | 0.05 |
| foam | acid/ | | | |
| | citrate | | | |
| Polyurethane | Citric | 0.10 | 1.51 | 0.02 |
| foam | acid/ | | | |
| | citrate | | | |
| Polyurethane | Benzoic | 0.02 | 0.16 | 0.01 |
| foam | acid/ | | | |
| | benzoate | | | |
| Polyurethane | Benzoic | 0.04 | 0.33 | 0.02 |
| foam | acid/ | | | |
| | benzoate | | | |
| Polyurethane | Lactic | 0.05 | 0.30 | 0.01 |
| foam | acid/ | | | |
| | lactate | | | |
| Polyurethane | Lactic | 0.10 | 0.62 | 0.05 |
| foam | acid/ | | | |
| | lactate | | | |
| Hydrogel | Citric | 0.05 | 0.13 | 0.01 |
| | acid/ | | | |
| | citrate | | | |
| Hydrogel | Lactic | 0.10 | 0.11 | 0.01 |
| | acid/ | | | |
| | lactate | | | |

Further measurements were carried out to determine how rapidly the buffer substances can dissolve in demineralized water from the foam layers and hydrogels. To this end, a 1 g sample of the foam layer or of the hydrogel of the wound care product according to the invention was placed in 15 ml of demineralized water and the water bath containing the wound care product was shaken at room temperature (25° C.) on a shaker at 100 mpm (shaking movements per minute). For each series of measurements, four samples were shaken, each in one water bath. The series of measurements was first carried out on foam layers.

In the case of the first sample, the buffering capacity of the liquid which contained buffer substances was measured in the first water bath after one hour by means of titration with NaOH. It was found that at least 90% of the maximum buffering capacity measurable after 24 hours had developed in the case of the first sample, i.e., as early as after one hour. Therefore, more than 90% of the buffer substances dissolvable in the liquid from the foam were dissolved in the liquid after one hour.

In the case of the second sample in the second water bath, the titration was carried out after 3 hours. The buffering capacity was over 90% of the maximum buffering capacity measurable after 24 hours.

In the case of the third sample in the third water bath, the titration was carried out after 6 hours. The buffering capacity was likewise over 90% of the maximum buffering capacity measurable after 24 hours.

In the case of the fourth sample in the fourth water bath, the titration was carried out after 24 hours. The buffering capacity determined in this measurement was set as 100% of the maximum buffering capacity measurable after 24 hours.

In the context of the series of measurements on foam layers, it was found that the buffering capacity in the measurements after 1, 3 and 6 hours remained constant on a plateau having over 90% of the maximum buffering capacity measurable after 24 hours.

In the case of the gels admixed with buffer substances, titration experiments were carried out analogously. After three hours, complete dissolution of the buffer substances from the gel in the liquid was established, the buffering capacity also remaining constant in the case of the third and fourth samples, i.e., after 6 hours and 24 hours.

1. A method for the treatment of a wound, said method comprising applying a wound care product to the wound, said wound care product comprising buffer substances and a wound dressing, the wound dressing comprising a foam, wherein the pH of a buffer solution resulting upon dissolution of the buffer substances in demineralized water at 37° C. is between pH 3 and pH 7.

2. The method of claim 1, wherein the pH of the buffer solution resulting upon dissolution of the buffer substances in demineralized water at 37° C. is between pH 3.5 and pH 6.

3. The method of claim 1, wherein the pH of the buffer solution resulting upon dissolution of the buffer substances in demineralized water at 37° C. is between pH 3.8 and pH 5.

4. The method of claim 1, wherein the wound care product comprises a wound contact layer comprising a hydrogel, a polymer film, a hydrocolloid matrix, a polymer network, a nonwoven and/or an adhesive.

5. The method of claim 1, wherein the wound care product comprises a hydrogel.

6. The method of claim 5, wherein the buffer substances are provided in the foam and/or in the hydrogel.

7. The method of claim 6, wherein the buffer substances are distributed homogeneously in the foam.

8. The method of claim 6, wherein the buffering capacity of the foam is, at a concentration of the buffer substances of 0.05 M, at least 0.25 mol of NaOH, more particularly at least 0.55 mol of NaOH.

9. The method of claim 6, wherein the buffering capacity of the hydrogel is, at a concentration of the buffer substances of 0.05 M, at least 0.08 mol of NaOH, more particularly at least 0.1 mol of NaOH.

10. The method of claim 1, wherein the buffer substances comprise citric acid and citrate as buffer substances.

11. The method of claim 1, wherein the buffer substances comprise lactic acid and lactate.

12. The method of claim 1, wherein the buffer substances comprise benzoic acid and benzoate.

13. The method of claim 1, wherein the buffering capacity of the wound care product reaches 90% of the maximum buffering capacity measurable after 24 hours after no later than three hours.

14. The method of claim 1 wherein the wound is a chronic wound.

15. The method of claim 1, wherein the wound care product comprises a layer comprising an open-pore, hydrophilic polyurethane foam.

16. The method of claim 1, further comprising microdebridement.

17. The method of claim 1, wherein the wound is in the inflammatory phase or granulation phase of wound healing.

18. A method for the treatment of a wound, said method comprising the steps of:
a. providing a foam,
b. providing a buffer solution containing buffer substances, the pH of the buffer solution at 37° C. being between pH 3 and pH 7,
c. impregnating the foam with the buffer solution,
d. completely or partially drying the foam
e. providing further layers,
f. joining the foam and the further layers to form a multilayered wound dressing, and
g. applying the wound dressing to the wound.

19. The method of claim 18 wherein at least one of the further layers comprises a hydrogel.

20. A method for the treatment of a wound, said method comprising the steps of:
a. providing a gel,
b. providing a wound dressing comprising a foam,
c. providing a buffer solution containing buffer substances, the pH of the buffer solution at 37° C. being between pH 3 and pH 7,
d. mixing the gel with the buffer solution,
e. joining the gel and the wound dressing to form a multilayered wound dressing, and
f. applying the wound dressing to the wound.
